(19) United States
(12) Patent Application Publication
(10) Pub. No.: US 2011/0144231 A1
(43) Pub. Date: Jun. 16, 2011

(54) Polyalkenoate Cements Having Improved Properties

(76) Inventors: Juergen Engelbrecht, Hamburg (DE); Ademola Olaseni Akinmade, Koelln-Reisiek (DE)

(21) Appl. No.: 13/000,691

(22) PCT Filed: Jun. 30, 2009
(86) PCT No.: PCT/EP2009/004699

§ 371 Date: Feb. 1, 2011

(30) Foreign Application Priority Data
Jun. 30, 2008 (DE) 10 2008 030 326.7

(51) Int. Cl. A61K 6/083; C08K 3/10; C08K 3/32

(52) U.S. Cl. 523/116

(57) ABSTRACT

The invention relates to a polyalkenoate cement for biomedical or dental applications, comprising: a) at least one acidic, phosphorus-based polyalkenoate polymer, b) at least one acid-soluble salt or acid-soluble compound of a multivalent metal, c) at least one non-polymeric acidic phosphorus compound and d) an ion-releasing, finely divided glass which is capable of reacting with components (a), (b) and (c) in the presence of water.

### FIELD OF THE INVENTION

The invention relates to a polyalkenoate cement having improved properties for biomedical or dental applications.

### PRIOR ART

Polyalkenoate cements have been known since 1972. The adhesive strength of those polyacid-based cements results from polycarboxylic acids which are capable of forming bonded complexes with both the apatite of teeth and the glass of the cement by way of their Ca ions. The following formula shows, in diagrammatic form, the crosslinking of polyacrylic acid by means of Ca²⁺:

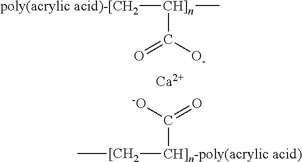

During the hardening phase of glass and acid, the cations (Ca²⁺, Al³⁺) leached out from the glass crosslink the chains of the polyacids. The result is the formation of a water-insoluble polysalt matrix in which there are embedded the partially reacted glass particles, which react further to form the cement.

These cements adhere well to the dentin and enamel of the tooth and exhibit good sealing properties when used as filling material in cavities. They prevent the ingress of liquids and bacteria and so protect against secondary caries. However, these cements are, prior to completion of the setting process, susceptible to moisture and, in addition, are acid-soluble. Also, the optical properties (translucency) do not meet current aesthetic requirements.

In order to further improve the properties of the described cements, Wilson tried in the late 80s of the last century to develop a new class of cements based on polyvinylphosphonic acids (poly(VPA)). The following formula shows polyVPA in diagrammatic form:

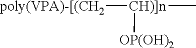

Aqueous solutions of poly(VPA) have a pH of about 1.5. Poly(VPA) is accordingly a stronger acid than polyacrylic acid and other polycarboxylic acids and so allows the preparation of cements that can be subjected to greater mechanical loading, as well as having the adhesive properties of the cements described hereinbefore.

Copolymers of vinylphosphonic acids with acrylic acid or other carboxylic acids can also be used in the preparation of such cements.

Such cements of glass and poly(VPA) are referred to as glass polyphosphonate cements. The first publications which describe cements comprising acid-attacked filler materials such as glass and phosphonic acids were presented by A. D. Wilson (EP 0340016 B1) and Ellis (EP 0431740 B1).

Disadvantageously, however, the setting behaviour of those cements was far too rapid for use in practice, because of the high acid strength of the poly(VPA). It is accordingly described in EP 0340016 B1 that the setting time of a cement prepared using polyacrylic acid, when replaced with poly(PVA), is reduced from about 3-4 minutes to 45 seconds, without even taking into account that the cement also has to be mixed and processed.

Akinmade and Braybrook were able to show in EP 0626842 that the setting times of poly(VPA)-based cements can be extended if the poly(VPA) is partially neutralised beforehand. Although this represented a further development, the still excessively rapid setting remained a disadvantage of those cements because the incorporation of a sufficiently large amount of powder, which should result in better mechanical properties compared to polycarboxylic acid-based cements, was not possible.

In 1995, Wilde and Williams, in patent specification UK 2291060, presented solely polycarboxylic acid-based glass ionomer cements which contained small amounts of copolymers of poly(PVA) in order to improve the mechanical properties. Because those cements were but very slightly modified versions of the cements known from the prior art, it was not surprising that the physical properties of those cements were only insignificantly different from those of the cements known beforehand.

The original objective—a cement based mainly or entirely on poly(VPA) in order to overcome the mentioned disadvantages such as, for example, slow setting, high acid-solubility or high opacity and the associated poor aesthetics of polycarboxylic acid-based glass ionomer cements—has not been achieved hitherto.

All polyalkane polymers having phosphorus-containing acidic groups or similarly structured polymers will be referred to hereinbelow as “acidic, phosphorus-based polyalkenoate polymers”. The cements formed by acid-base reaction when those polymers are used in the preparation will be referred to as “phosphorus-based polyalkenoate cements”.

Hitherto there have been no polyalkenoate cements usable or on the market wherein the polyalkenoate acids entirely or partly comprise phosphorus-containing acidic groups.

### PROBLEM OF THE INVENTION

The problem of the invention was to obtain a phosphorus-based polyalkenoate cement that can be applied well in practice and that in respect of the properties important for that purpose—such as a sufficient processing time along with a rapid hardening time, lower solubility, good mechanical loadability and also better aesthetics—is far superior to the conventional above-mentioned polyalkenoate cements based on polycarboxylic acids.

It has been possible, surprisingly, to solve the problem of the invention as a result of the fact that new polyalkenoate cements have been found which are suitable for biomedical and/or dental applications and which contain the following components (a) to (d):
(a) at least one acidic, phosphorus-based polyalkenoate polymer and
(b) at least one acid-soluble salt or acid-soluble compound of a multivalent metal and
(c) at least one non-polymeric, acidic phosphorus compound and
(d) an ion-releasing, finely divided glass which is capable of reacting with components (a), (b) and (c) in the presence of water.

Components (a) to (c) can be mixed with the ion-releasing, finely divided glass (component (d)), especially in the presence of water, and they react to form a cement.

In fully hardening, cements according to the invention, containing the combination of (b) and (c), exhibit a sufficient processing time that is convenient in use, improved setting characteristics, improved mechanical properties such as, for example, greater hardness or less abrasion and even optical properties that are further improved.

In a preferred embodiment according to the invention, the compositions according to the invention comprise (i) an acid solution comprising components (a), (b) and (c) and also (ii) an acid-soluble glass (component (d)). The acid solution (liquid) accordingly contains, in this embodiment, components (a), (b) and (c). By this means, within a short period after the processing time has ended, the multivalent cations (also referred to herein as “rapidly available multivalent cations”) are available for the setting reaction, which contrasts with the cements known from the prior art in which several hours go by before complete availability of the multivalent cations from the glass powder.

In a further preferred embodiment according to the invention, component (b) can be admixed with the powdered glass (component (d)) in dry form. The liquid accordingly contains, in this embodiment, components (a) and (c).

In a further preferred embodiment according to the invention, the acidic, phosphorus-based polyalkenoate polymer (component (a)) can be admixed with the powdered glass (component (d)) in dry form. The liquid accordingly contains, in this embodiment, components (b) and (c).

In a further preferred embodiment according to the invention, both the acidic, phosphorus-based polyalkenoate polymer (component (a)) and the acid-soluble salt (component (b)) can be admixed with the powdered glass (component (d)) in dry form. The liquid accordingly contains, in this embodiment, only component (c).

Also possible are embodiments in which components (a) and/or (b) in dry form are admixed with component (d) and also contained in dissolved form in the liquid.

Surprisingly, the combination of “rapidly available multivalent cations” with a non-polymeric, acidic phosphorus component results in delay of the cement setting reaction together with good mechanical values, which, in contrast to the cements known from the prior art, allows processability and, consequently, excellent usability of those cements.

The combination of (a) and (b) alone with (d) or the combination of (a) and (c) alone with (d) does not result in the described improved properties, which are achieved only with the combination of (a), (b) and (c) with (d).

### MORE DETAILED DESCRIPTION OF THE INVENTION

Unless expressly defined otherwise, percentages signify percentages by weight. In the case of the liquid which may contain components (a), (b) and/or (c), preference is given to the remainder consisting of water.

The acidic, phosphorus-based polyalkenoate polymers (component (a)) may be homopolymers of vinylphosphonic acid, homopolymers of vinylphosphoric acid and also copolymers of vinylphosphonic acid and/or polymers of unsaturated phosphoric acids which are optionally copolymerised with unsaturated carboxylic acids. These mentioned polyacids may also be used in partially esterified form. Also suitable are vinylphosphonic acid esterified with unsaturated carboxylic acids, vinylphosphonic acid esterified with unsaturated phosphonic acids, and vinylphosphonic acid esterified with unsaturated phosphoric acids.

Suitable monomers for synthesis of the desired acidic phosphorus-based polyalkenoate polymers or copolymers may be, for example, vinylphosphonic acid, α-methyl-vinylphosphonic acid, styrene vinyl phosphonic acid, acrylic acid, 2-chloro-acrylic acid, 3-chloroacrylic acid, 2-bromoacrylic acid, 3-bromoacrylic acid, methacrylic acid, itaconic acid, maleic acid, glutaric acid, citraconic acid, mesaconic acid, fumaric acid and tiglic acid.

Further suitable monomers for copolymerisation with the mentioned unsaturated phosphorus-based acids may be, for example, acrylamide, acrylonitrile, vinyl chloride, allyl chloride, vinyl acetate and 2-hydroxyethyl methacrylate.

Component (a) preferably comprises a copolymer of polyvinylphosphonic acid with unsaturated carboxylic acids, especially with maleic acid, acrylic acid, itaconic acid or combinations thereof.

If the liquid which may contain components (a), (b) and/or (c) does contain component (a), the amount of component (a) in the liquid is preferably from 10 to 70% by weight, more preferably from 20 to 60% by weight, and even more preferably from 30 to 50% by weight.

The acid-soluble salt or the acid-soluble compound of a multiple-valency (i.e. multivalent) metal cation (component (b)) must be soluble in an aqueous solution of the acidic, phosphorus-based polyalkenoate polymer in the presence of (c) without precipitate formation. Suitable salts or compounds for this combination may be, for example, those having divalent cations such as Ca²⁺, Sr²⁺, Ba²⁺, Ce²⁺, TiO²⁺, ZrO²⁺, Fe²⁺, Co²⁺, Cu²⁺, Zn²⁺ or, preferably, trivalent cations such as Sc³⁺, Y³⁺, La³⁺, Yb³⁺, Cr³⁺, Mo³⁺, W³⁺, Fe³⁺, Ru³⁺, Os³⁺, Au³⁺, Al³⁺, Ga³⁺, In³⁺.

Very special preference is given to those having Al³⁺ or Fe³⁺ cations.

Anions of the acid-soluble salt or of the acid-soluble compound of a multivalent metal may be, for example, O²⁻, [CO₃]²⁻, Cl⁻, [SO₄]²⁻, [PO₄]³⁻, [HPO₄]²⁻, [H₂PO₄]⁻, [HPO₂]²⁻, [H₂PO₃]⁻.

Suitable constituents of component (b) are, for example, Al₂(SO₄)₃, AlCl₃, FeCl₃ and AlH₆(PO₄)₃. Special preference is given to using the metal salts in the form of dihydrogen phosphate salts, which may at the same time also constitute a constituent of component (c), with AlH₆(PO₄)₂ being especially preferred in that case.

If the liquid which may contain components (a), (b) and/or (c) does contain component (b), the amount of component (b) in the liquid is preferably 0.1-50% by weight, more preferably 0.1-10% by weight and even more preferably 0.1-5% by weight.

The non-polymeric acidic phosphorus compound (component (c)) may be organic-inorganic or purely inorganic in nature. It may be phosphoric acid or a phosphoric acid derivative; it may be a phosphonic acid or a phosphonic acid derivative. Acidic phosphates and acidic phosphonates or combinations with the above are likewise suitable. Acid esters of those phosphorus-containing monomers are also suitable.

Preferred examples are monophosphonic acids, diphosphonic acids or ortho-phosphoric acid, with diphosphonic acids and ortho-phosphoric acid being especially preferred. Suitable examples of acidic phosphates are dihydrogen phosphates and organic acidic phosphates of formula (RO)P(O) (OH)₂ and (RO)₂P(O) (OH), where R=e.g. butyl, n-octyl, n-hexyl etc.

Preferred examples of acidic phosphonates are bis(chloroethyl)vinyl phosphonates.

The amount of component (c) in the liquid which may contain components (a), (b) and/or (c) is preferably 0.1-50% by weight, more preferably 5-30% by weight and even more preferably 10-20% by weight.

Suitable ion-releasing, finely divided glasses (component (d)) which may react with components (a), (b) and (c) in the presence of water are ion-releasing glasses from the prior art such as, for example, divalent-metal-cation-doped aluminosilicate glasses and melts which comprise fluoride, alkali metal ions and/or phosphates. These glasses are reactive and are preferably characterised by an SiO₂/Al₂O₃ ratio of 0.6-5.0:1, especially 2.0-5.0:1.

The glass may be a purely synthetic glass or may include a natural mineral which, for example, reacts with an acid first by way of a gel, as is described, for example, in EP 0 883 586 B1. A preferred example of a synthetic glass is an aluminosilicate glass which may be produced, for example, by melting together quartz (SiO₂) and alumina (Al₂O₃), optionally with addition of one or more suitable fluoride, carbonate and/or phosphate salt(s) such as, for example, CaF₂, CaCO₃, AlPO₄, Ca₃PO₄ and the like. Synthetic glasses of such a kind are well-known in the field and are described, for example, by A. D. Wilson et al. “Aluminosilicate Glasses for Polyelectrolyte Cements” I&EC Product Research & Development, Vol. 19, pp. 263-270, 1980. A glass as understood by this invention may furthermore also include a so-called “non-fused oxyfluoride material” which in addition to a trivalent metal ion (preferably aluminium), oxygen ions and fluoride ions also includes alkaline earth metal ions such as, for example, strontium, calcium and barium. Examples of preferred glasses are known in the field and are described, for example, in the publications EP 0 883 566 B1 and WO 2007/017152 A2. They may be purchased in suitable form from customary commercial sources.

The expression “may react in the presence of water” is understood to be, especially, a release of ions, primarily cations, from the glass, which ultimately results in full hardening of the cement.

The expression “finely divided” means that the glasses have a particle diameter with a D₉₉ value of less than 100 μm, preferably less than 30 μm and more preferably less than 20 μm.

The particle diameter is conventionally given as the average particle diameter (D₅₀ and/or D₉₉ value) and can be determined, for example, using a “Sediment Analyser” measuring apparatus. The D₅₀ value of the particle diameter of the finely divided glass particles is preferably from 0.5 to 20 μm, more preferably 5 μm or less and especially from 0.5 to 5.0 μm. The D₉₉ value of the particle diameter of the finely divided glass particles is preferably from 2 to 40 μm, more preferably 30 μm or less and especially from 2 to 30 μm.

The ratio of the glass (component (d)) to the liquid which may contain components (a), (b) and/or (c) is preferably from 5:1 to 0.5:1 and more preferably from 3:1 to 0.8:1. If the polyalkenoate cement according to the invention is to be used as a biomedical or dental filling material a relatively high proportion of glass has been found to be advantageous, with a preferred ratio of glass to liquid of from 4.0:1 to 2.0:1, and especially from 3.0:1 to 2.0:1. If the polyalkenoate cement according to the invention is to be used as a biomedical or dental fixing material, a relatively low proportion of glass has been found to be advantageous, with a preferred ratio of glass to liquid of from 1:1 to 0.6:1, especially of about 0.8:1.

Component (d) may additionally comprise a water-soluble or at least partially water-soluble multivalent metal cation compound, a sufficiently high amount of which is dissolved during the initial hardening phase of the mixed cement. The expression “at least partially water-soluble” means here that at least 2 g, preferably at least 4 g, of the multivalent metal cation compound can be dissolved in 100 mL of water at room temperature. The expression “a sufficiently high proportion” means here that at least at least 30%, preferably at least 60%, more preferably at least 90%, of the multivalent metal cation compound is dissolved during the initial hardening phase of the mixed cement. The expression “initial hardening phase” refers here, for example, to a period of 0-10 mins., preferably of 0-5 mins, after mixing of component (d) with the other components. In particular applications, the initial hardening phase may also be a period of 0-30 mins. or even 0-60 mins.

Use of the cements according to the invention is feasible for various indications in the biomedical and dental field. Preference is given to use as an aesthetic dental restoration and fixing material.

The cements may be present in powder/liquid form, in which case the liquid is an aqueous solution of the acidic components. In another embodiment, the acidic components may also be present, in part or in entirety, in the powder in substantially dry form. The components of the cements according to the invention may also be present in paste form, in which case otherwise customarily used paste-formers such as thickeners, humectants and also resins—especially hydrophilic resins—are incorporated with the appropriate components of this invention.

The cement according to the invention may further comprise one or more customary additives and adjuvants such as, for example, suitable indicators, colourants, pigments, inhibitors, accelerators, viscosity-modifying agents, wetting agents, surface-active agents, buffering agents, stabilising agents, chelating agents and the like, which are known in the field. If desired, the cement may also comprise one or more medicaments or therapeutic substances such as fluoridation agents, anti-caries agents (for example, xylitol), remineralisation agents (for example, calcium phosphate compounds), contrast agents and the like, which are customarily used in the field in dental cements.

### EXAMPLES

### I. Tests on Setting Behaviour and Mechanical Properties

The following Examples are intended to illustrate the above arrangements.

For preparation of the cements, the liquids (see below for compositions) are admixed with an ion-releasing finely divided glass (EonGlass, Benco company, United States) in a ratio of glass to liquid of 2.4:1.

Components of liquids used:

a.) acidic, phosphorus-based polyalkenoate polymer
a₁: 40% aqueous solution of a copolymer of poly(vinylphosphonic acid) and acrylic acid, pH=0.7
   b.) acid-soluble salt of a multivalent metal
b₁: Al₂ (SO₄)₃
b₂: AlCl₃
b₃: FeCl₃
b₄: AlH₆ (PO₄)₃
   c.) non-polymeric, acidic phosphorus compound
c₁: H₃PO₄
c₂: diphosphonic acid

TABLE 1
Compositions (proportions by weight) of cements

| | a₁ | b₁ | b₂ | b₃ | b₄ | c₁ | c₂ |
|---|---|---|---|---|---|---|---|
| Ex. | PVPA | Al₂(SO₄)₃ | AlCl₃ | FeCl₃ | AlH₆(PO₄)₃ | H₃PO₄ | Diphosphonic acid |
| 1 | 100 | — | — | — | — | — | — |
| 2 | 100 | — | — | — | — | 10 | — |
| 3 | 100 | — | — | — | — | — | 10 |
| 4 | 100 | 4 | — | — | — | 10 | — |
| 5 | 100 | — | 4 | — | — | 10 | — |
| 6 | 100 | — | — | 4 | — | 10 | — |
| 7 | 100 | — | — | — | 4 | 10 | — |
| 8 | 100 | — | — | — | 8 | 10 | — |
| 9 | 100 | 4 | — | — | — | — | 10 |
| 10 | 100 | — | 4 | — | — | — | 10 |
| 11 | 100 | — | — | 4 | — | — | 10 |
| 12 | 100 | — | — | — | 4 | — | 10 |
| 13 | 100 | — | — | — | 12 | — | 10 |
| 14 | 100 | — | — | — | 24 | — | 10 |
| 15 | 100 | 4 | — | — | — | — | — |
| 16 | 100 | — | 4 | — | — | — | — |
| 17 | 100 | — | — | 4 | — | — | — |
| 18 | 100 | — | — | — | 4 | — | — |

TABLE 2
Properties of the liquids and the cements

| | Appearance | Processing | Setting | Bending | Compressive | In accordance |
|---|---|---|---|---|---|---|
| | of the | time | time | strength | strength | with the |
| Ex. | liquid | [min.] | [min.] | [MPa.] | [MPa] | invention? |
| 1 | clear | X | X | X | X | No |
| 2 | clear | 0:42 | 2:17 | 18 | 98 | No |
| 3 | clear | 0:30 | 1:20 | X | X | No |
| 4 | clear | 1:16 | 2:38 | 32 | 157 | Yes |
| 5 | clear | 1:15 | 2:30 | 32 | 161 | Yes |
| 6 | clear | 1:22 | 2:45 | 38 | 184 | Yes |
| 7 | clear | 1:35 | 3:00 | 42 | 200 | Yes |
| 8 | clear | 2:25 | 3:40 | 40 | 195 | Yes |
| 9 | clear | 1:24 | 1:50 | 33 | 128 | Yes |
| 10 | clear | 1:28 | 1:55 | 30 | 148 | Yes |
| 11 | clear | 1:20 | 1:52 | 43 | 210 | Yes |
| 12 | clear | 1:45 | 2:20 | 42 | 205 | Yes |
| 13 | clear | 3:10 | 4:40 | 36 | 191 | Yes |
| 14 | clear | 7:40 | 12:10 | 32 | 182 | Yes |
| 15 | Y | Z | Z | Z | Z | No |
| 16 | Y | Z | Z | Z | Z | No |
| 17 | Y | Z | Z | Z | Z | No |
| 18 | Y | Z | Z | Z | Z | No |

X = setting too rapid, so cannot be measured
Y = non-homogeneous, cloudy, in some cases with precipitate
Z = not possible to mix a homogeneous cement

Tables 1 and 2 show that both component (b) and component (c) are necessary for improvement of the physical properties. Omission of (b) results in setting that is too rapid and in cements that are mechanically too weak, whereas without component (c) only cloudy and non-homogeneous liquids are obtained, which makes it impossible to mix a homogeneous cement.

It is possible to adjust the processing time almost at will by means of the amount of (b) added (in the presence of (c)).

1. Polyalkenoate cement for biomedical or dental applications, comprising
(a) at least one acidic, phosphorus-based polyalkenoate polymer,
(b) at least one acid-soluble salt or acid-soluble compound of a multivalent metal,
(c) at least one non-polymeric, acidic phosphorus compound and
(d) an ion-releasing, finely divided glass which is capable of reacting with components (a), (b) and (c) in the presence of water.

2. Polyalkenoate cement according to claim 1, characterised in that component (a) includes a vinylphosphonic acid or a copolymer of vinylphosphonic acid, or a vinylphosphonic acid esterified with unsaturated carboxylic acids, or a vinylphosphonic acid esterified with unsaturated phosphonic acids, or a vinylphosphonic acid esterified with unsaturated phosphoric acids.

3. Polyalkenoate cement according to claim 1, characterised in that the multivalent metal is a divalent metal cation.

4. Polyalkenoate cement according to claim 3, characterised in that the divalent metal cation is Ca²⁺, Sr²⁺, Ba²⁺, Ce²⁺, TiO²⁺, ZrO²⁺, Fe²⁺, Co²⁺, Cu²⁺ and/or Zn²⁺.

5. Polyalkenoate cement according to claim 1, characterised in that the multivalent metal is a trivalent metal cation.

6. Polyalkenoate cement according to claim 5, characterised in that the trivalent metal cation is Sc³⁺, Y³⁺, La³⁺, Yb³⁺, Cr³⁺, Mo³⁺, W³⁺, Fe³⁺, Ru³⁺, Os³⁺, Au³⁺, Al³⁺, Ga³⁺ and/or In³⁺.

7. Polyalkenoate cement according to claim 1, characterised in that component (b) is present in an amount of 0.1-50% by weight, based on the entirety of the liquid which may contain components (a), (b) and/or (c).

8. Polyalkenoate cement according to claim 1, characterised in that component (b) is present in an amount of 0.1-10% by weight, based on the entirety of the liquid which may contain components (a), (b) and/or (c).

9. Polyalkenoate cement according to claim 1, characterised in that component (b) is present in an amount of 0.1-5% by weight, based on the entirety of the liquid which may contain components (a), (b) and/or (c).

10. Polyalkenoate cement according to claim 1, characterised in that component (d) comprises a water-soluble or at least partially water-soluble multivalent metal cation compound, a sufficiently high proportion of which is dissolved during the initial hardening phase of the mixed cement.

11. Polyalkenoate cement according to claim 1, characterised in that component (c) comprises phosphoric acid, acidic phosphates, phosphonic acid, acidic phosphonates or mixtures thereof.

12. Polyalkenoate cement according to claim 11, characterised in that the phosphoric acid is di(ethylhexyl)phosphoric acid or ortho-phosphoric acid or partially neutralised ortho-phosphoric acid.

13. Polyalkenoate cement according to claim 11, characterised in that the acidic phosphate is aluminium hexahydrogen triphosphate and/or calcium tetrahydrogen diphosphate.

14. Polyalkenoate cement according to claim 11, characterised in that the phosphonic acid is monophosphonic acid or a diphosphonic acid.

15. Polyalkenoate cement according to claim 1, characterised in that component (c) is present in an amount of 0.1-50% by weight, based on the entirety of the liquid which may contain components (a), (b) and/or (c).

16. Polyalkenoate cement according to claim 1, characterised in that component (c) is present in an amount of 5-30% by weight, based on the entirety of the liquid which may contain components (a), (b) and/or (c).

17. Polyalkenoate cement according to claim 1, characterised in that component (c) is present in an amount of 10-20% by weight, based on the entirety of the liquid which may contain components (a), (b) and/or (c).
